(19) United States
(12) Patent Application Publication
(10) Pub. No.: US 2004/0006243 A1
(43) Pub. Date: Jan. 8, 2004

(54) Method of producing oxybutynin and its derivatives

(75) Inventors: Masakatsu Shibasaki, Mitaka City (JP); Motomu Kanai, Bunkyo-Ku (JP)

Correspondence Address: OLIFF & BERRIDGE, PLC, P.O. BOX 19928, ALEXANDRIA, VA, 22320, US

(73) Assignee: THE UNIVERSITY OF TOKYO, Bunkyo-Ku (JP)

(21) Appl. No.: 10/438,851

(22) Filed: May 16, 2003

(30) Foreign Application Priority Data
May 30, 2002 (JP) 2002-157,290

(51) Int. Cl. C07C 229/34; C07F 9/80

(52) U.S. Cl. 560/58; 549/207; 549/218

(57) ABSTRACT

An oxybutynin and its derivatives are produced by reacting a phenylketone with a silylcyanide in the presence of a specified asymmetric catalyst to obtain a siloxynitrile, and then reacting the siloxynitrile with a reducing agent and oxidizing the resulting aldehyde, or subjecting the siloxynitrile to a hydrolysis.

### BACKGROUND OF THE INVENTION

1. Field of the Invention

The present invention relates to a method of producing an oxybutynin and its derivatives, and more particularly to a method of producing an oxybutynin and its derivatives using an asymmetric catalyst.

2. Description of Related Art

The oxybutynin and its derivatives (Ditropan, trade mark of oxybutynin chloride) are applicable as a bronchodilator or a remedy for pollakisuria, and are less in the side effect, and are drugs more increasing their level of importance in an aged society. They are an antagonist for a muscarine receptor, and rest in phase 3 of the clinical test stage as a lead of the remedy for the pollakisuria.

As a synthesis of such a useful oxybutynin, there is known a diastereoselective synthesis method using an equivalent amount of a chiral modification agent.

However, the synthesis method using the chiral modification agent has a problem that it is impossible to provide the oxybutynin in a high environmental harmony because it is necessary to conduct desorption of an equivalent weight of a chiral source.

Therefore, the feature that it is possible to provide such useful oxybutynin and derivatives thereof in a high environmental harmony will be very high in the contribution to medicine and pharmaceutics in future.

### SUMMARY OF THE INVENTION

It is, therefore, an object of the invention to provide a method capable of commonly producing an oxybutynin and its derivatives in large quantities.

In order to achieve the above objects, the inventors have made various studies with respect to catalytic asymmetric cyanosilylation reaction of aldehyde or imine and the like, and found out a method of producing an oxybutynin and its derivatives according to the invention.

According to the invention, there is the provision of a method of producing an oxybutynin and its derivatives, which comprises reacting a phenylketone with a silylcyanide in the presence of an asymmetric catalyst formed by bonding a metal to a catechol portion of a ligand represented by the following general formula (I):

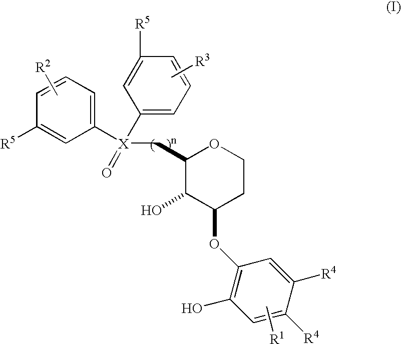

(wherein each of R¹, R² and R³ is a substituent on an aromatic ring, and R⁴ is a hydrogen atom or an electron attractive group, provided that a ring-closing structure may be formed by two R⁴, and R⁵ is a methyl group, a methoxy group, a dimethyl amino group or an electron attractive group, and X is P or As, and n is 1 to 3) to form a siloxynitrile, and then reacting the siloxynitrile with a reducing agent to obtain an aldehyde and oxidizing the aldehyde, or subjecting the siloxynitrile to a hydrolysis.

In a preferable embodiment of the invention, the phenylketone is at least one selected from the group consisting of cyclohexyl phenylketone, cyclopentyl phenylketone or its fluorine-substituted derivative, cyclobutyl phenylketone and derivatives substituted on a phenyl group thereof.

In another preferable embodiment of the invention, the metal is bonded as a metal complex.

In the other preferable embodiment of the invention, the metal complex has a structure represented by the following formula (II):

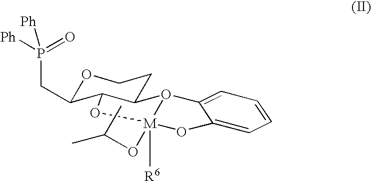

(wherein M is a metal, and R⁶ is a nonexistent state, or an alkoxide, CN, Cl, F, Br or I).

In a further preferable embodiment of the invention, the metal is at least one selected from the group consisting of titanium, zirconium, ytterbium, aluminum, gallium, gadolinium, samarium and lanthanum.

In a still further preferable embodiment of the invention, the metal is a rare earth metal.

In a yet further preferable embodiment of the invention, the reducing agent is at least one selected from the group consisting of diisobutylaluminum hydride, Raney nickel, lithium triethylborohydride (Superhydride), and diisopropylaluminum hydride.

### DETAILED DESCRIPTION OF THE INVENTION

The ligand used in the invention is represented by the formula (I):

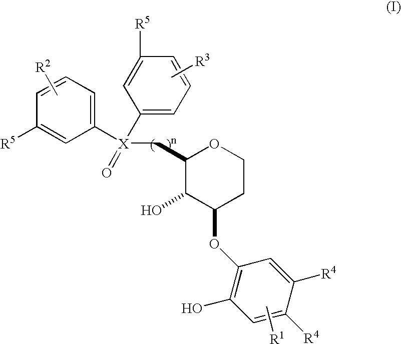

(wherein each of R¹, R² and R³ is a substituent on an aromatic ring, and R⁴ is a hydrogen atom or an electron withdrawing group, provided that a ring-closing structure may be formed by two R⁴, and R⁵ is a methyl group, a methoxy group, a dimethyl amino group or an electron withdrawing group, and X is P or As, and n is 1 to 3). Moreover, the catalysis may be carried out by using plural ligands having different values of n in the formula (I).

A ligand constituting the skeleton of the formula (I) can be synthesized, for example, according to the following reaction formulae:

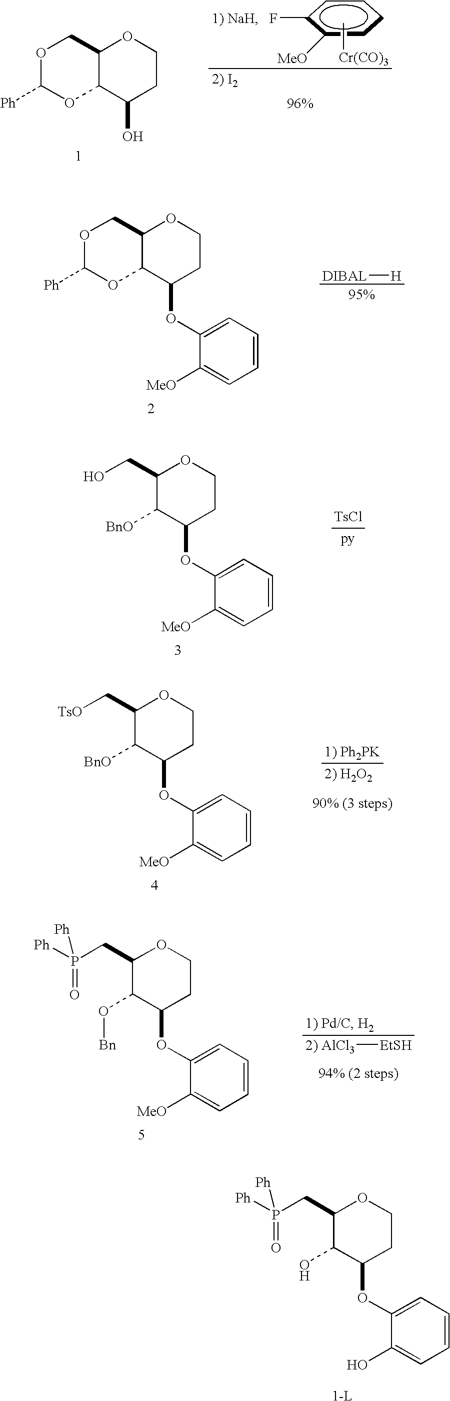

An alcohol 1 is rendered into a sodium alkoxide and subjected to a nucleophilic displacement reaction with an arene chromium complex to obtain an acetal 2 in which a catechol portion is introduced into the hydroxyl group of the alcohol 1. The alcohol used as a starting material is not particularly limited, but may include, for example, alcohols starting from sugar. The acetal 2 is reduced with DIBAL-H to obtain a compound 3, which is subjected to a tosylation to obtain a compound 4. The compound 4 is reacted with Ph₂PK and oxidized with H₂O₂ to obtain a compound 5. The compound 5 is subjected to a reductive debenzylation with a palladium (Pd/C) catalyst and then deblocking of methyl ether is conducted with AlCl₃-EtSH, whereby a ligand 1-L can be obtained.

As shown in the above reaction formulae, the ligand 1-L can be easily synthesized from an alcohol on a scale of about 5 g.

In the formula (I), each of R¹, R² and R³ is not particularly limited, but is a substituent on an aromatic ring. As the substituent, mention may be made of an alkyl group, an ether group, an amine group, an ester group and so on. The group R¹ is preferable to be an ester group from a viewpoint of an enhancement of Lewis acidity, while the groups R², R³ are preferable to be an ether group, an amine group or an alkyl group from a viewpoint of an enhancement of Lewis basicity.

As R⁴ can be mentioned an electron attractive group other than a hydrogen atom. As the electron attractive group, mention may be made of —F, —NH₃, —Cl, —CF₃, —CCl₃, —NO₂, —CN, —CHO₄, —COCH₃, —CO₂H, —SO₂CH₃, benzoyl group and a benzoyl analog from a viewpoint of a stronger attraction.

As R⁵ are mentioned a hydrogen atom, a methyl group, a methoxy group, a dimethyl amino group and an electron attractive group. Such an electron attractive group may be the same as used in the group R⁴.

The asymmetric catalyst used in the invention is formed by bonding a metal to a catechol portion of the ligand of the formula (I). The asymmetric catalyst means a catalyst having an ability of producing an optically active material in itself, i.e. an enantio-differentiating catalyst. The metal is possible to form a metal complex at a hydroxyl group of the catechol portion of the ligand.

As the metal to be bonded to the catechol portion can be mentioned at least one selected from the group consisting of titanium, zirconium, ytterbium, aluminum and gallium. These metals may be used alone or in a combination of two or more. As the metal, titanium is preferable from a viewpoint of a high enantio-selectivity.

A rare earth metal can be mentioned as the metal bonded to the catechol portion. As the rare earth metal can be mentioned at least one selected from the group consisting of La, Ce, Pr, Nd, Pm, Sm, Eu, Gd, Dy, Ho and Er. Among them, Gd and Sm are preferable as the rare earth metal from a viewpoint of a high enantio-selectivity.

In the asymmetric catalyst used in the invention, the metal complex has a structure represented by the formula (II):

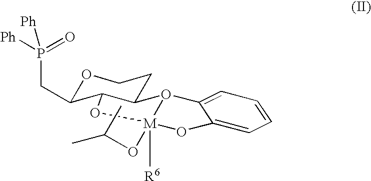

Also, in the asymmetric catalyst used in the invention, wherein the metal complex is generated using the ligand and the metal alkoxide in a ratio of 1:1 to 1:3, preferably, in a ratio of 2:1.

In case of titanium, zirconium and the like may be taken the structure of the formula (II). As R⁶ may be mentioned an alkoxide, CN, Cl, F, Br and I. By using the alkoxide, CN, Cl, F, Br or I as the group R⁶ can be stabilized the asymmetric catalyst. On the other hand, ytterbium or the like as the metal may not require the group R⁶ such as CN or the like in view of the bonding conformation.

The asymmetric catalyst used in the invention can act as a catalyst for a cyanosilylation reaction of a ketone. The cyanosilylation reaction means that a nucleophilic addition of a cyanide is taken to a carbonyl carbon and the resulting alkoxide is caught by a silyl group.

According to the method of the invention, oxybutynin and its derivatives can be obtained by reacting a ketone with a silylcyanide in the presence of the above asymmetric catalyst to obtain a siloxynitrile and then reacting the siloxynitrile with a reducing agent to obtain an aldehyde and thereafter oxidizing the aldehyde.

The siloxynitrile obtained by the cyanosilylation reaction of the ketone makes it possible to provide useful materials such as quaternary α-hydroxy carboxylic acid and the like at one step.

The ketone used as a substrate for the asymmetric catalyst according to the invention is a phenylketone. As the phenylketone, mention may be made of cyclohexyl phenylketone, cyclopentyl phenylketone or a fluorine-substituted derivative, cyclobutyl phenylketone and derivatives substituted on a phenyl group thereof.

As the silylcyanide, mention may be made of trimethyl silylcyanide (TMSCN), triethyl silylcyanide, t-butyldimethyl silylcyanide and so on. Moreover, HCN, trimethyl tin cyanide and the like may be mentioned as a material capable of providing siloxynitrile in the same manner other than silylcyanide.

Further, a solvent used in the cyanosilylation reaction of ketone is not particularly limited. As the solvent, mention may be made of low polar solvents such as toluene, CH₂Cl₂ and the like; coordination solvents such as tetrahydrofuran (THF), dimethoxyethane, ether, acetonitrile and propionitrile, and so on. The coordination solvents such as tetrahydrofuran (THF), dimethoxyethane, ether, acetonitrile and propionitrile are preferable as the solvent from a viewpoint of increasing a reaction rate and providing a high enantio-selectivity.

A temperature of the cyanosilylation reaction may be a room temperature and is not particularly limited, but is preferable to be from −78° C. to the room temperature, more preferably from −60° C. to 0° C., particularly from −60° C. to −20° C. The reason why the lower limit is −78° C. is based on the enhancement of the enantio-selectivity, while the reason why the upper limit is the room temperature is based on the increase of the reaction rate.

Moreover, a concentration of the ketone is not particularly limited and may be properly changed in accordance with the product to be targeted. As the ketone concentration becomes higher, the reaction rate tends to become high.

An aldehyde is obtained by reacting the thus obtained siloxynitrile with a proper reducing agent. Thereafter, oxybutynin and its derivatives can finally be obtained by oxidizing the aldehyde.

As the reducing agent, mention may be made of diisobutyl aluminum hydride, Raney nickel, Superhydride, diisopropylalcohol aluminum hydride. Among them, diisobutyl aluminum hydride is preferable from a viewpoint of a high yield.

In this case, the reaction temperature may be a room temperature and is not particularly limited, but is preferable to be from −100° C. to 20° C., more preferably from −78° C. to −40° C. from a viewpoint of a high yield. The reason why the lower limit is −100° C. is based on the prevention of a side reaction, while the reason why the upper limit is the room temperature is based on the increase of the reaction rate.

That is, when the siloxynitrile is reduced with diisobutyl aluminum hydride, the reduction is completed by dissolving or suspending the siloxynitrile in a solvent such as CH₂Cl₂, toluene, hexane or the like, adding 1 to 5 equivalent weight of diisobutyl aluminum hydride to the siloxynitrile and stirring them at a proper temperature between −78° C. and −40° C. for 1-24 hours. Thereafter, an aldehyde is obtained by conducting a post treatment, if necessary.

The thus obtained aldehyde is oxidized with a proper oxidizing agent such as sodium chlorite, potassium permanganate, potassium bichromate or the like, whereby an oxybutynin and its derivatives can be obtained.

Alternatively, oxybutynin and its derivatives can be obtained by directly subjecting the siloxynitrile to a hydrolysis.

The following examples are given in illustration of the invention and are not intended as limitations thereof. Moreover, it should be understood that changes and modifications may easily be made without any departure from the spirits of the invention.

### EXAMPLE 1

There is first examined a ligand 1-L in which R¹ to R³ in the formula (I) are at nonexistent state and each of R⁴ and R⁵ is a hydrogen atom.

[3-benzyloxy-4-(2-methoxyphenyl)-tetrahydro-pyrano[3,2-d][1,3]dioxyn-8-al alcohol (is rendered into sodium alkoxide and subjected to a nucleophilic displacement reaction with an arene chromium complex to obtain 8-(2-methoxyphenyl)-2-phenyl-hexahydro-pyrano[3,2-d][1,3]dioxyn (hereinafter referred to as compound 2) in which a catechol portion is introduced into a hydroxyl group of the alcohol. The compound 2 is reduced with DIBAL-H to form [3-benzyloxy-4-(2-methoxyphenyl)-tetrahydro-pyran-2-yl]methanol (hereinafter referred to as compound 3). The compound 3 is subjected to a tosylation to obtain toluene-4-sulfonic acid 3-benzyloxy-4-(2-methoxyphenyl)-tetrahydro-pyran-2-yl-methylester (hereinafter referred to as compound 4). The compound 4 is reacted with Ph₂PK and oxidized with H₂O₂ to form 3-benzyloxy-2-(diphenyl phosphinoylmethyl)-4-(2-methoxyphenyl)-tetrahydro-pyran (hereinafter referred to as compound 5). The compound 5 is subjected to a reduction debenzylation with palladium (Pd/C) catalyst and further to deblocking of methyl ether with AlC13-EtSH to obtain a ligand 1-L.

The values of physical properties of the thus obtained ligand 1-L are shown below.

Melting point: 219-220° C.

¹H-NMR (500 MHz. CDCl3) δ1.94 (m, 1H), 2.14 (m, 1H), 2.69 (ddd, J=9.8, 15.0, 15.0 Hz, 1H), 2.84 (ddd, J=2.8, 9.5, 15.3 Hz, 1H), 3.23 (ddd, J=1.9, 12.2, 12.2 Hz, 1H), 3.34 (dddd, J=2.8, 7.0, 9.4, 9.8 Hz, 1H), 3.55 (ddd, J=5.5, 8.9, 11.6 Hz, 1H), 3.73 (dd, J=8.9, 9.4 Hz, 1H), 3.90(ddd, J=1.2, 5.7, 12.2 Hz, 1H), 6.71 (ddd, J=1.9, 7.4, 7.4 Hz, 1H), 6.96 (m, 3H), 7.51 (m, 6H), 7.75 (m, 4H), 8.92 (s, 1H); ¹³C-NMR (125 MHz, CDCl₃) δ31.62, 37.61(d, J=68 Hz), 65.50, 74.96, 76.11, 84.84, 117.22, 119.14, 122.45, 125.50, 128.90, 129.00, 129.03, 129.13, 130.60(d, J=10 Hz), 131.11(d, J=9 Hz), 132.47, 145.89, 150.15; 31P- NMR (202 MHz, CDCl₃), δ34.0

IR 3422, 1156, 1103 cm⁻¹

Analytical value as C₂₅H₂₇O₅P: C, 67.67; H, 6.10%. 02070 (2002-157,290)

Found value: C, 67.92; H, 5.94%

Next, it is tried to synthesize an oxybutynin derivative by using the above ligand. The synthesis root is shown as follows.

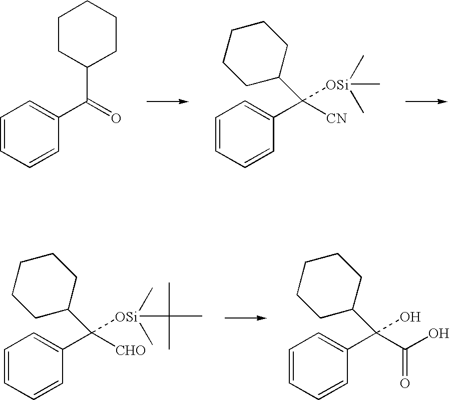

A commercially available cyclohexyl phenylketone is subjected to a cyanosilylation at −60° C. or −40° C. using 5 or 1 mol % of (S)-selective catalyst prepared by mixing Gd(OiPr)3 and the ligand 1-L at a mixing ratio of 1:2 for 32 hours. After the completion of the reaction, an objective cyanohydrin is obtained in a yield of 96-100% and an enantiomer excess of 94%ee. In this case, TMS(tetramethylsilane)CN (120 mol %) is used as a silylcyanide, and propionitrile is used as a solvent.

The spectrum data of (S)-cyanohydrin are shown as follows:

1H-NMR 0.09 (s, 9H), 1.02-1.21 (m, 5H), 1.35-1.39 (m, 1H), 1.62-1.65 (m, 1H), 1.68-1.75 (m, 2H), 1.79-1.82 (m, 1H), 1.99-2.03 (m, 1H), 7.32-7.39 (m, 3H), 7,45-7.47 (m, 2H)

Then, the cyanohydrin is reduced with diisobutyl aluminum hydride (in CH₂Cl₂ solvent, −78° C., 8 hours) and oxidized with sodium chlorite to obtain a basic skeleton of oxybutynin in a yield of 36-68%.

### EXAMPLE 2

There are examined a ligand 2 in which each of R¹-R³ and R⁵ in the formula (I) is a hydrogen atom and R⁴ is a fluorine atom, and a ligand 3 in which each of R¹-R³ and R⁵ in the formula (I) is a hydrogen atom and R⁴ takes a closed ring structure. The same experiment as in Example 1 is carried out to try the production of a basic skeleton of an oxybutynin. The results are shown in Table 1.

TABLE 1

| | | Concen- | | | | |
|---|---|---|---|---|---|---|
| | | tration of | Reaction | Re- | | |
| | | asymmetric | tempera- | action | | |
| | Metal:ligand | catalyst | ture | time | Yield | |
| Ligand | (mol ratio) | (mol %) | (° C.) | (h) | (%) | ee/% |
| Ligand | 1:2 | 5 | −60 | 21 | 96 | 95 |
| 1 | 1:2 | 2 | −60 | 96 | 98 | 84 |
| | 1:2 | 1 | −60 | 216 | 39 | 64 |
| | 1:2 | 1 | −40 | 40 | 100 | 94 |
| Ligand | 1:2 | 5 | −60 | 18 | 98 | 96 |
| 2 | | | | | | |
| Ligand | 1:2 | 5 | −60 | 18 | 93 | 95 |
| 3 | | | | | | |

In Table 1, ee represents an enantiomer excess. As seen from Table 1, the ligand 1 attains excellent yield and enantiomer excess even when the concentration of the asymmetric catalyst is properly changed. Similarly, the good results are obtained even in the ligands 2 and 3.

The basic skeleton of oxybutynin is prepared by using these ligands in the same manner as in Example 1, respectively. As a result, any skeletons are obtained in a yield of 36-68%.

The method of producing an oxybutynin and its derivatives according to the invention develops an advantageous effect that important medical goods having high general-purpose properties can be provided in large quantities and pharmaceutical products at a high versatility with a high environmental harmony. Therefore, the invention largely contributes to the study of medicine and pharmaceutical sciences.

What is claimed is:

1. A method of producing an oxybutynin and its derivatives, which comprises reacting a phenylketone with a silylcyanide in the presence of an asymmetric catalyst formed by bonding a metal to a catechol portion of a ligand represented by the following general formula (I): 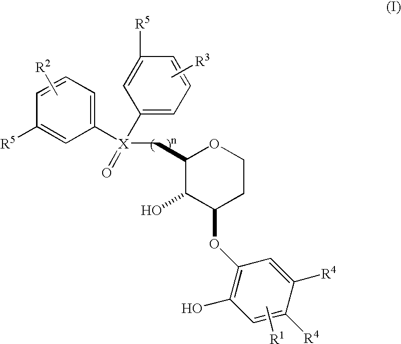
(wherein each of R¹, R² and R³ is a substituent on an aromatic ring, and R⁴ is a hydrogen atom or an electron attractive group, provided that a ring-closing structure may be formed by two R⁴, and R⁵ is a methyl group, a methoxy group, a dimethyl amino group or an electron attractive group, and X is P or As, and n is 1 to 3) to form a siloxynitrile, and then reacting the siloxynitrile with a reducing agent to obtain an aldehyde and oxidizing the aldehyde, or subjecting the siloxynitrile to a hydrolysis.

2. The method according to claim 1, wherein the phenylketone is at least one selected from the group consisting of cyclohexyl phenylketone, cyclopentyl phenylketone or its fluorine-substituted derivative, cyclobutyl phenylketone and derivatives substituted on a phenyl group thereof.

3. The method according to claim 1, wherein the metal is bonded as a metal complex.

4. The method according to claim 3, wherein the metal complex has a structure represented by the following formula (II): 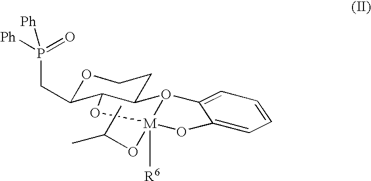
(wherein M is a metal, and R⁶ is a nonexistent state, or an alkoxide, CN, Cl, F, Br or I).

5. The method according to claim 3 or 4, wherein the metal is at least one selected from the group consisting of titanium, zirconium, ytterbium, aluminum, gallium and a rare earth metal.

6. The method according to claim 5, wherein the rare earth metal is gadolinium, samarium or lanthanum.

7. The method according to claim 1, wherein the reducing agent is at least one selected from the group consisting of diisobutylaluminum hydride, Raney nickel, Superhydride and diisopropylaluminum hydride.

8. The method according to claim 3, wherein the metal complex has a structure containing the metal and ligand as mentioned in claim 1 in a ratio of 1:1 to 1:3.
